(19) United States
(12) Patent Application Publication
(10) Pub. No.: US 2024/0409919 A1
(43) Pub. Date: Dec. 12, 2024

(54) METHOD FOR SEPARATING COMPONENTS USING SUPERCRITICAL FLUID CHROMATOGRAPH

(71) Applicants: SHIMADZU CORPORATION, Kyoto (JP); OSAKA UNIVERSITY, Osaka (JP)

(72) Inventors: Satoshi OBIKA, Suita-shi (JP); Takao YAMAGUCHI, Suita-shi (JP); Momoka HAYASHIDA, Kyoto-shi (JP); Junichi MASUDA, Kyoto-shi (JP); Shinosuke HORIE, Duisburg (DE); Risa SUZUKI, Kyoto-shi (JP)

(73) Assignees: SHIMADZU CORPORATION, Kyoto (JP); OSAKA UNIVERSITY, Osaka (JP)

(21) Appl. No.: 18/733,430

(22) Filed: Jun. 4, 2024

(30) Foreign Application Priority Data
Jun. 8, 2023 (JP) 2023-094936

(51) Int. Cl. C12N 15/10; B01D 15/40

(52) U.S. Cl. CPC C12N 15/101; B01D 15/40

(57) ABSTRACT

A method for separating components using a supercritical fluid chromatograph, including: injecting a sample into a mobile phase containing a supercritical fluid and a modifier to introduce the sample into a column; and separating components in the sample during passing through the column; wherein the sample contains an oligonucleotide as a target component; the supercritical fluid contains carbon dioxide; and the modifier contains a solution containing at least one selected from the group consisting of ammonium, an alkylamine, and an amino alcohol, and an acid.

### CROSS REFERENCE TO RELATED APPLICATIONS

This nonprovisional application is based on Japanese Patent Application No. 2023-094936 filed on Jun. 8, 2023, with the Japan Patent Office, the entire contents of which are hereby incorporated by reference.

### BACKGROUND OF THE INVENTION

### Field of the Invention

The present invention relates to a method for separating components using a supercritical fluid chromatograph.

### Description of the Background Art

The supercritical fluid chromatograph (hereinafter also referred to as “SFC”) is a chromatograph that uses a fluid (supercritical fluid) having a temperature and pressure exceeding a critical point (critical temperature, critical pressure) as a main mobile phase, and has a feature that an elution performance can be controlled by adjusting the temperature and pressure of the mobile phase. In SFC, carbon dioxide is widely used as a supercritical fluid because of its relatively low critical point in both temperature and pressure, easy handling, and low cost.

Vincent Desfontaine et al. Journal of Chromatography A, 1562, 96-107, 2018, discloses a method for analyzing human metabolomics using SFC. WO 2016/152996 discloses a stationary phase of SFC with good molecular distinction.

### SUMMARY OF THE INVENTION

In recent years, a liquid chromatograph (hereinafter also referred to as “LC”) and a high performance liquid chromatograph (hereinafter also referred to as “HPLC”) have been used to analyze an oligonucleotide, but there is a case where a separation characteristic may not be sufficient. For example, in a nucleic acid therapeutic which is an oligonucleotide, a phosphorothioate linkage is sometimes used instead of a phosphodiester linkage of the oligonucleotide to improve stability in a body. In a synthetic process of the oligonucleotide containing the phosphorothioate linkage, there is a case where an analogue is produced that is partially converted to the phosphodiester linkage. In LC and HPLC, it is difficult to distinguish one to a few tens of atomic differences in the oligonucleotide of several thousand molecular weight and to separate a major component from the analogue.

An object of the present invention is to provide a method for separating components using a supercritical fluid chromatograph capable of separating an oligonucleotide well.

As a result of their intensive research, the present inventors have found that the oligonucleotide could be successfully separated in the supercritical fluid chromatograph by using a prescribed modifier, and completed the present invention.

A first aspect of the present invention relates to a method for separating components using a supercritical fluid chromatograph, including: injecting a sample into a mobile phase containing a supercritical fluid and a modifier to introduce the sample into a column; and separating components in the sample during passing through the column; wherein the sample contains an oligonucleotide as a target component; the supercritical fluid contains carbon dioxide; and the modifier includes a solution containing at least one selected from the group consisting of ammonium, an alkylamine, and an amino alcohol, and an acid.

The above and other objects, features, aspects, and advantages of the present invention will become apparent from the following detailed description of the invention as understood in conjunction with the drawings attached.

### BRIEF DESCRIPTION OF THE DRAWINGS

FIG. 1 shows a flow path configuration that illustrates an example of an SFC performing a component separation method with respect to the present embodiment;

FIG. 2 shows a chromatogram from the analysis of Example 1;

FIG. 3 shows a chromatogram from the analysis of Example 2;

FIG. 4 shows a chromatogram from the analysis of Example 3;

FIG. 5 shows a chromatogram from the analysis of Example 4;

FIG. 6 shows a chromatogram from the analysis of Example 5;

FIG. 7 shows a chromatogram from the analysis of Example 6;

FIG. 8 shows a chromatogram from the analysis of Comparative Example 1; and

FIG. 9 shows a chromatogram from the analysis of Comparative Example 2.

### DESCRIPTION OF THE PREFERRED EMBODIMENTS

The present invention is a method for separating components using a supercritical fluid chromatograph (SFC), including: injecting a sample into a mobile phase containing a supercritical fluid and a modifier to introduce the sample into a column; and separating components in the sample during passing through the column; wherein the sample contains an oligonucleotide as a target component; the supercritical fluid contains carbon dioxide (CO₂); and the modifier contains a solution containing at least one selected from the group consisting of ammonium, an alkylamine, and an amino alcohol, and an acid. The separation method of the present invention can be used in an analytical method for analyzing components of a sample. Hereinafter, one embodiment of the component separation method with respect to the present invention will be described.

### [SFC]

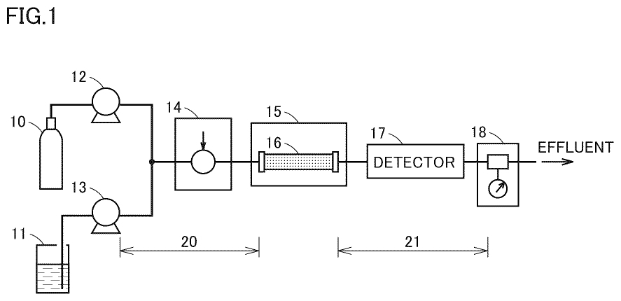

FIG. 1 shows a flow path configuration that illustrates an example of an SFC performing a component separation method with respect to the present embodiment. The SFC shown in FIG. 1 is equipped with a liquefied CO₂ cylinder 10, a modifier tank 11, a CO₂ pump 12, a liquid delivery pump 13, an autosampler 14, a column oven 15, a column 16, a detector 17, and a back-pressure regulator 18. Liquefied CO₂ cylinder 10 is a source of liquefied CO₂ as the supercritical fluid. Modifier tank 11 is a tank that pools the characteristic modifier described later, which is added to the supercritical fluid.

The basic operation of this SFC is as follows. Back-pressure regulator 18 adjusts a pressure of the mobile phase in an inlet side channel 20 between CO₂ pump 12 and liquid delivery pump 13 and an inlet of column 16, in column 16, and in an outlet side channel 21 between an outlet of column 16 and back-pressure regulator 18 to a pressure above the critical pressure of CO₂ (7.4 MPa), for example 10 MPa. CO₂ pump 12 draws the liquefied CO₂ from liquefied CO₂ cylinder 10 and delivers it to inlet side channel 20. Meanwhile, liquid delivery pump 13 draws the modifier from modifier tank 11 and delivers it to inlet side channel 20. The liquefied CO₂ and the modifier are mixed in inlet side channel 20 and delivered to column 16 as the mobile phase. Autosampler 14 provided in inlet side channel 20 injects a liquid sample into the mobile phase at a predetermined time.

Column oven 15, in which column 16 is installed, is adjusted to a temperature above the critical temperature (31° C.) of CO₂ which is a major substance of the mobile phase, e.g., about 35° C. Therefore, the mobile phase passing through column 16 becomes supercritical state. The sample introduced into column 16 with flow of the mobile phase is separated into individual components by interaction with a stationary phase provided on an inner wall of column 16. Then, the individual components elute from column 16 with a time difference and flow into outlet side channel 21. Detector 17 provided in outlet side channel 21 is a UV-visible spectroscopic detector (UV detector) or the like, which detects the components in the mobile phase separated in column 16 keeping a critical state. The mobile phase after component detection is collected through back-pressure regulator 18 into a collecting vessel or the like, not shown in the drawing.

As detector 17, any of optical detectors such as a multi-wavelength detector using a PDA (Photo Diode Array) used in a liquid chromatograph, an optical rotation detector, a circular dichroism detector, a fluorescence detector, a refractive index detector, and an evaporative light scattering detector may be used. A mass spectrometer using an atmospheric pressure ion source, such as an electrospray ion source, may also be used as detector 17.

### [Mobile Phase]

The mobile phase includes the supercritical fluid containing carbon dioxide and the modifier. The modifier contains at least one selected from the group consisting of ammonium, an alkylamine, and an amino alcohol (hereinafter also referred to as a component (a)), and an acid (hereinafter also referred to as a component (b)). The modifier further contains a solvent. The modifier can improve a separation performance of the target component, an oligonucleotide, by including component (a) and component (b).

Examples of alkylamine include ethylamine and methylamine. Examples of amino alcohol include aminoethanol and aminomethanol. Examples of acid include formic acid, acetic acid, and bicarbonate. In the modifier, sources of component (a) and component (b) may be the same or different. For example, component (a) and component (b) may be provided simultaneously by adding ammonium formate, ammonium acetate, or ammonium bicarbonate. Examples of solvent include various organic solvents and water. Examples of various organic solvents include methanol, ethanol, isopropyl alcohol, and acetonitrile.

In the modifier, each concentration of component (a) and component (b) is preferably 10 mM or more and 100 mM or less, more preferably 40 mM or more and 50 mM or less. By being within the above numerical range, the separation performance of the target component, the oligonucleotide, can be further improved. In the modifier, a mixture of water and methanol is preferably used as the solvent, and the mixed solvent, for example, water: methanol of 5:95 (volume ratio) can be used.

### [Stationary Phase]

The stationary phase is not limited as long as it is one commonly used in SFC. For example, silica gel, a chemically modified silica support surface, and a polymer bead can be used. An example of a suitable stationary phase is a silica support surface modified with an alkyl alcohol.

### [Sample to be Separated]

The sample to be separated contains an oligonucleotide as the target component. The oligonucleotide is an oligomer of which a nucleotide is a constituent unit, preferably 2 to 40 nucleobases, more preferably 2 to 20 nucleobases, and even more preferably 2 to 10 nucleobases in length. The nucleotide may be either a ribonucleotide, a deoxyribonucleotide, or a modified nucleotide. The modified nucleotide is one in which all or a part of the nucleobase, a sugar moiety, and a phosphate linkage moiety constituting the ribonucleotide or deoxyribonucleotide are modified.

Examples of nucleobase include adenine, guanine, cytosine, thymine, uracil, and modified bases thereof. Such modified bases include, but are not limited to, a 5-alkylcytosine (e.g., 5-methylcytosine).

Examples of modification of the sugar moiety include modification of the 2′-position of a ribose and modifications of other parts of the sugar. Examples of modification of the 2′-position of the ribose include replacement of an-OH group at the 2′-position of the ribose with OR, R′OR and F, wherein R represents alkyl; and R′ represents alkylene. Examples of modification of other parts of the sugar include, but are not limited to, substitution of O to S at the 4′ position of the ribose or deoxyribose, cross-linking of the 2′ and 4′ positions of the sugar, e.g., LNA (Locked Nucleic Acid) or ENA (2′-O,4′-C-Ethylene-bridged Nucleic Acids).

Examples of modification of the phosphate linkage moiety include modifications that replace a phosphodiester linkage with a phosphorothioate linkage, a phosphorodithioate linkage, an alkylphosphonate linkage, a phosphoramidate linkage, and a boranophosphate linkage.

According to the separation method of the present embodiment, when the sample to be separated contains two or more oligonucleotides, the two or more oligonucleotides can be separated well, whether a difference in molecular structure between the two or more oligonucleotides is a difference of one to several tens of atoms, or one or more and ten or less atoms. Therefore, according to the separation method of the present embodiment, the target component can be separated or purified with high accuracy even when the sample to be separated is a sample containing an oligonucleotide which is the target component and an oligonucleotide different from the target component which was generated in the synthetic process of the target component.

Examples of samples to be separated include an oligonucleotide containing a base sequence TAGC, and an oligonucleotide containing a base sequence TTTT.

### EXAMPLES

Hereinafter, the present invention will be described in more detail with reference to examples, but not limited to the following examples.

### [Samples to be Analyzed]

Samples containing eight oligonucleotides (components a to h) shown in Table 1 were prepared.

TABLE 1

| Component | Base Sequence |
|---|---|
| Component a | 5′-TTTT-3′ |
| Component b | 5′-T*TTT-3′ |
| Component c | 5′-T*TT*T-3′ |
| Component d | 5′-T*T*T*T-3′ |
| Component e | 5′-TAGC-3′ |
| Component f | 5′-T*AGC-3′ |
| Component g | 5′-T*AG*C-3′ |
| Component h | 5′-T*A*G*C-3′ |

In Table 1, T: deoxythymidine, A: deoxyadenine, G: deoxyguanine, C: deoxycytidine, *indicates that the phosphodiester linkage in the phosphate linkage moiety of the nucleotide on either side is the phosphorothioate linkage.

### [Separation Column]

A column for SFC (Shin-pack UC-Diol II, produced by Shimadzu Corporation) (100 mm×2.0 mm I.D., 3 μm) filled with a silica filler modified with an alkyl alcohol was used.

### Examples 1 to 6, Comparative Examples 1, 2

The above separation column was attached to a supercritical fluid chromatogram system (Nexera UC, produced by Shimadzu Corporation), and analyses of the above samples to be analyzed were performed by SFC-MS/MS. The analysis conditions were as follows.
Mobile phase: mobile phase A (CO₂): mobile phase B (modifier listed in Table 2)=50:50 (volume ratio)
Flow rate of mobile phase: 1.5 mL/min
Temperature of column oven: 35° C.
Pressure of back-pressure valve: 10.0 MPa (pressure at 50° C.)
Sample injection volume: 5 μL
Detector: photodiode array detector (260 nm)

TABLE 2

| | Component | Component | |
|---|---|---|---|
| | (a) | (b) | Solvent |
| Example 1 | 50 mM | 50 mM | water:methanol = 5:95 |
| | methylamine | acetic acid | |
| Example 2 | 50 mM | 50 mM | water:methanol = 5:95 |
| | ethylamine | acetic acid | |
| Example 3 | 50 mM | 50 mM | water:methanol = 5:95 |
| | aminoethanol | acetic acid | |
| Example 4 | 50 mM ammonium formate | | water:methanol = 5:95 |
| Example 5 | 50 mM ammonium acetate | | water:methanol = 5:95 |
| Example 6 | 50 mM ammonium bicarbonate | | water:methanol = 5:95 |
| Compar- | — | 50 mM | water:methanol = 5:95 |
| ative | | formic acid | |
| Example 1 | | | |
| Compar- | — | 0.1% (v/v) | water:methanol = 5:95 |
| ative | | trifluoroacetic acid | |
| Example 2 | | | |

In Table 2, water:methanol = 5:95 represents the volume ratio.

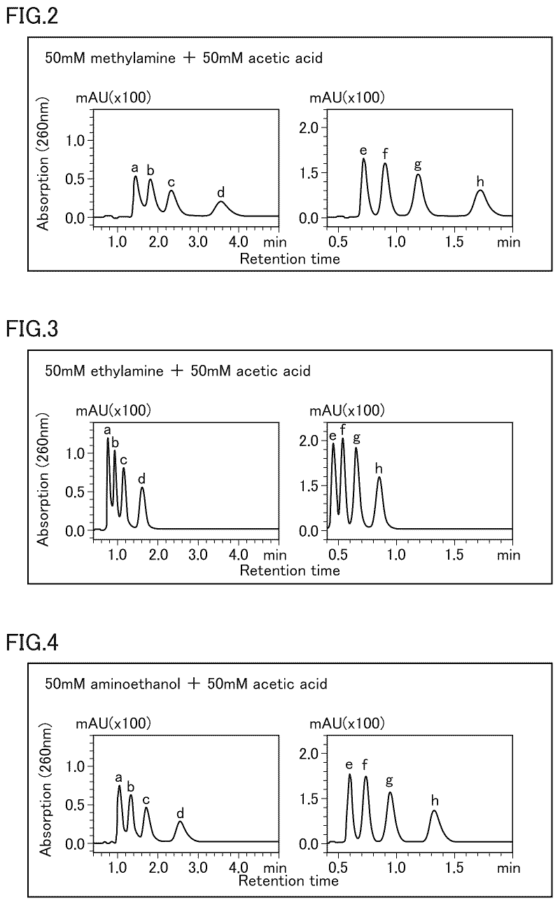

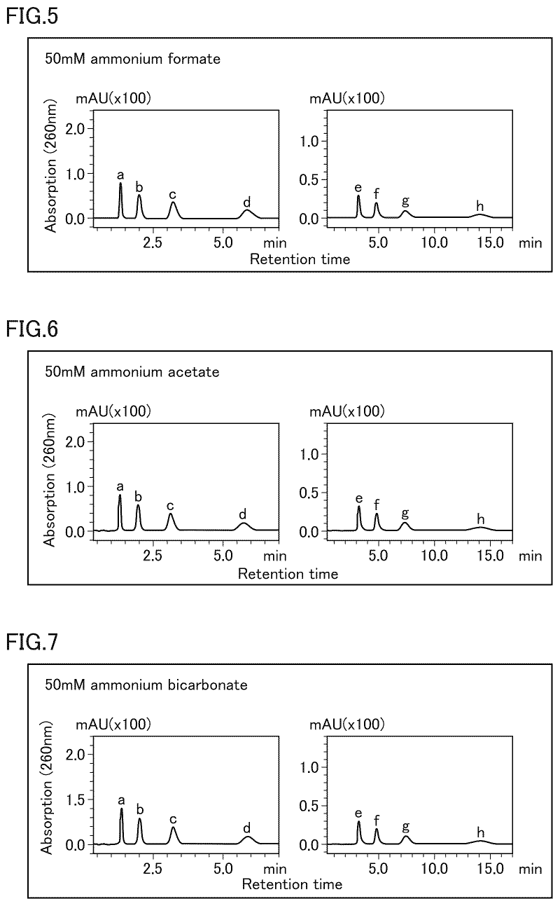

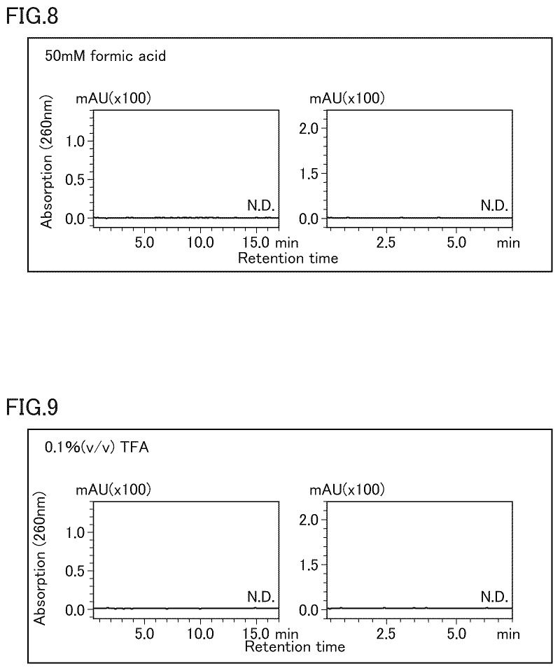

FIGS. 2 to 9 show chromatograms obtained by the analyses of Examples 1 to 6 and Comparative Examples 1 and 2. In FIGS. 2 to 9, peaks a to h correspond to components a to h, respectively. The results shown in FIGS. 2 to 9 indicate that components a to h could be separated in Examples 1 to 6, whereas components a to h could not be separated in Comparative Examples 1 and 2.

### Aspects

It is understood by a person skilled in the art that a plurality of exemplary embodiments and examples described above are specific examples of the following aspects.

(Paragraph 1) The method for separating components using a supercritical fluid chromatograph with respect to one aspect is
a method for separating components using a supercritical fluid chromatograph, including: injecting a sample into a mobile phase containing a supercritical fluid and a modifier to introduce the sample into a column; and separating components in the sample during passing through the column; wherein the sample contains an oligonucleotide as a target component; the supercritical fluid contains carbon dioxide; and the modifier contains a solution containing at least one selected from the group consisting of ammonium, an alkylamine, and an amino alcohol, and an acid.

(Paragraph 2) The method for separating components according to paragraph 1, wherein the acid is at least one selected from the group consisting of formic acid, acetic acid, and bicarbonate.

(Paragraph 3) The method for separating components according to paragraph 1 or 2, wherein the oligonucleotide contains a base sequence TAGC.

(Paragraph 4) The method for separating components according to any of paragraphs 1 to 3, wherein the oligonucleotide has replaced at least one of phosphodiester linkages in a phosphate linkage moiety with a phosphorothioate linkage.

(Paragraph 5) The method for separating components according to any of paragraphs 1 to 4, the column has a stationary phase containing an alkyl alcohol.

Although the embodiments of the present invention have been described, the embodiments disclosed herein should be considered in all respects to be illustrative and not restrictive. The scope of the invention is indicated by the claims and is intended to include all modifications within the meaning and scope of the claims and equivalents.

What is claimed is:

1. A method for separating components using a supercritical fluid chromatograph, comprising:
injecting a sample into a mobile phase containing a supercritical fluid and a modifier to introduce the sample into a column; and
separating components in the sample during passing through the column; wherein
the sample comprises an oligonucleotide as a target component;
the supercritical fluid comprises carbon dioxide; and
the modifier comprises a solution containing at least one selected from the group consisting of ammonium, an alkylamine, and an amino alcohol, and an acid.

2. The method for separating components according to claim 1, wherein the acid is at least one selected from the group consisting of formic acid, acetic acid, and bicarbonate.

3. The method for separating components according to claim 1, wherein the oligonucleotide comprises a base sequence TAGC.

4. The method for separating components according to claim 1, wherein the oligonucleotide has replaced at least one of phosphodiester linkages in a phosphate linkage moiety with a phosphorothioate linkage.

5. The method for separating components according to claim 1, wherein the column has a stationary phase containing an alkyl alcohol.
